(19) United States
(12) Patent Application Publication
(10) Pub. No.: US 2018/0104346 A1
(43) Pub. Date: Apr. 19, 2018

(54) Vaginal Bioadhesive Boric Acid Formulation and Its Preparation Method

(71) Applicants: Yu Hui, Winston Salem, NC (US); Xudong Yuan, Morganville, NJ (US); Tian Zhang, Richmond (CA)

(72) Inventors: Yu Hui, Winston Salem, NC (US); Xudong Yuan, Morganville, NJ (US); Tian Zhang, Richmond (CA)

(73) Assignee: Xudong Yuan, Morganville, NJ (US)

(21) Appl. No.: 15/294,724

(22) Filed: Oct. 15, 2016

(51) Int. Cl. A61K 47/32; A61K 33/22; A61K 9/00

(52) U.S. Cl. CPC A61K 47/32; A61K 9/0034; A61K 33/22

(57) ABSTRACT

This invention discloses a bioadhesive boric acid formulation and its preparation method. Specifically, preferred vaginal bioadhesive boric acid formulation mainly comprises chitosan, carbopol 974 , polycarbophil, boric acid, and purified water as a gel. The main performance characteristic of this vaginal boric acid preparation is the ability of the formulation to retain and release boric acid in the vagina by using a combination of carbopol 974 and polycarbophil as preferred bioadhesive polymers. It was proved experimentally that boric acid as the antibacterial component of this gel can be maintained at a relatively high concentration (>10 mg/mL) in vagina for an extended period of time (>2 hours) with the use of a combination of polyacrylic acid polymers, i.e. carbopol 974 and polycarbophil. In order to prepare a gel of the said formulation with uniform continuity, boric acid was dissolved in water at the beginning of the process to make an aqueous solution and the bioadhesive polymers were added portion-wise into the aqueous solution with vigorous stirring. Further, it was demonstrated that the said vaginal bioadhesive boric acid formulation had a dynamic viscosity >100000 cps, pH value of 2 to 5, good moisture-retention capability. The formulation was non-allergenic, non-vaginal irritative, and capable of inhibiting bacteria and fungi.

### FIELD OF THE INVENTION

The present invention relates to a vaginal bioadhesive boric acid formulation and its preparation method. Vaginal bioadhesive boric acid formulation is a new formulation of boric acid. It uses bioadhesive polymer material as skeleton structure, retaining and releasing boric acid in vaginal fluid as antibacterial ingredient. The invention is related to pharmaceutics, analytical chemistry, and microbiology.

### PRIOR ART

About 80-95% of vaginitis originates from the infection of candida albicans. T. glabrata infections accounted for only 5% -15% of vaginitis, but the treatment options are very limited. There are only a few reports in the literature about the treatments of T. glabrata vaginitis.

However, the incidence of T. glabrata is constantly increasing. This is probably because of the overuses of systemic oral conazoles, short-term treatment regimens and non-prescription topical antifungal therapies.

Most of T. glabrata vaginitis are chronic and relapsing. These vaginal fungi are increasingly resistant to imidazole and conazoles. The lowered sensitivity of T. glabrata to these drugs may be the result of the selection effects of the antifungal agents for mutated T. glabrata.

It was reported that boric acid concentration at 10-20 mg/mL in urine could suppress almost all common fungi or bacteria.

In an in vitro study, 4% of boric acid inhibited the proliferation of 2×106 per mL of C. albicans. Although boric acid has not been widely studied as a treatment for fungal vaginitis, it was reported that boric acid treatment of vulvovaginal candidiasis resulted in a cure rate of >90%. Boric acid treatment for 14 days was equivalent to conazoles.

For these reasons, it was recommended that boric acid be used as first-line treatment of refractory T. glabrata vaginitis. Clinical efficacy of boric acid can reach as high as 81%, while mycological eradication rate can reach 77%.

The use of boric acid 600 mg per day for a therapeutic course of two weeks did not cause adverse reactions. But genital burning sensation in some patients was common. Recurrent fungal vaginitis, can be treated with boric acid capsules twice a week for 1-3 months. 600 mg capsule formulation is usually an extemporaneous preparation by pharmacy, because boric acid vaginal formulation has not been approved for marketing and the commercial products are not available for clinicians and patients.

Bacterial vaginosis is the most common vaginal infection. Although antibiotic therapy against anaerobic bacteria can effectively relieve short-term symptoms, recurrence rate of approximately 30% after 3 months of therapy is largely frustrating.

There are reports for antibiotic maintenance therapy, such as the use of 0.75% of vaginal metronidazole gel twice a week. Despite not completely curative, the therapy reduced the recurrence of bacterial vaginosis. Unfortunately, had patients stopped using this suppressive maintenance therapy, the bacterial vaginosis would relapse.

It was hypothesized that the formation of G. vaginalis biological biofilm on the vaginal epithelial wall antagonizes antibacterial effect of metronidazole. The topical application of boric acid for vaginal antimicrobial therapy can promote biofilm removal, help eradicate G. vaginalis, reduce the invasion of other potential bacterial pathogens, and therefore lessen the recurrence of bacterial vaginosis.

In one study, patients with a cumulative total of 60 recurrent bacterial vaginosis participated and completed boric acid therapy in addition to nitroimidazole. At week 7 and 12, the cure rate of the combination therapy were 88% and 92% respectively. At week 16, and 28, the cumulative cure rates were 78% and 65% respectively. No adverse reactions were observed for boric acid.

Due to the decline of estrogen levels, middle-aged women tend to show the symptoms of atrophic vaginitis. It is estimated that 10-40 percent of postmenopausal women have symptoms of atrophic vaginitis. Throughout their life cycle, women experience changes in vaginal epithelium influenced by circulating estrogen levels. In childhood, the vaginal epithelium is shallow. Upon puberty, the vaginal epithelium is thicken under estrogen stimulation. Estrogen also promotes the production of abundant glycogen, the substrate Lactobacillus uses to produce lactic acid, which lowers vaginal pH to 3.5˜4.5, forming a natural defensive barrier to prevent vaginal and urinary tract infections. The rise of vaginal pH makes vagina susceptible to Streptococcus, Staphylococcus, E coli, and other bacterial infections.

After menopause, estrogen (primarily estradiol) levels decrease from greater than 120 micrograms per milliliter to about 18 picograms per milliliter. Then there are plenty of cytological changes with the decline of hormone levels, including connective tissue proliferation, elastin disintegration, and glass-like changes of collagen. These changes may lead to granulation, fissures, bruising, telangiectasia and ulcers. Changes happen to not only postmenopausal genital tract, but also urethra. Both vaginal and urinary tract epithelial cells undergo unfavorable changes in postmenopausal women because of their estrogen dependence.

Menopause is the leading cause of the decrease of circulating estrogen levels. Therefore, it is the main reason of atrophic vaginitis. In premenopausal women, radiation therapy, chemotherapy, immune disorders and oophorectomy etc. can suppress ovarian estrogen production. Breastfeeding postpartum women, estrogen levels will also fall in breastfeeding women due to the antagonism of prolactin. The side effects of anti-estrogen drugs, including methylhydroxyprogesterone, tamoxifen, danazol, leuprolide and nafarelin, may also trigger atrophic vaginitis. The severity of atrophic vaginitis is related to the frequency of sexual intercourse, smoking or not, with or without vaginal delivery, whether the patients had vaginal surgery or not, estrogen levels prior to menopause and so on.

The premise of atrophic vaginitis is usually a long-term reduction in estrogen stimulation. A reduction in vaginal lubrication is an early marker of reduced estrogen. Genital Symptoms include skin dryness, burning sensation, pressure feeling, pain during intercourse, lack of vaginal secretions, increased vaginal discharge, and itching. Urinary tract symptoms include urinary discomfort, urinary urgency, hematuria, urinary tract infection, urination difficulties and stress incontinence. Atrophic vaginitis can be combined with Candida infection, trichomonas infection, or bacterial vaginosis. These infections can exacerbate the symptoms of vaginal atrophy. Over time, insufficient vaginal lubrication often leads to sexual dysfunction and emotional distress.

Because the lack of natural estrogen is the primary reason for the emergence of atrophic vaginitis, hormone replacement therapy is the most logical choice. Estrogen replacement therapy can restore normal pH, promote vaginal epithelial thickening and allow the regeneration of vaginal epithelial vessels. Suitable estrogen replacement therapy helps increase the number of superficial cells, alleviate the existing symptoms, and prevent the development of symptoms of the genitourinary system. But contraindications of estrogen therapy include estrogen-sensitive tumors, stage liver failure, and estrogen-dependent thrombosis. Adverse reactions of estrogen therapy are also very prominent, including breast tenderness, increased risk of vaginal bleeding, increased risk of estrogen-dependent tumors, and endometrial cancer and hyperplasia. Additionally, estrogen therapy typically has a long onset time. To completely eliminate dryness, 24 months of therapy may be necessary. Even so, some patients (approximately 10-25%) do not respond to this treatment.

Therefore, reluctance to use estrogen, estrogen therapy contraindications, or adverse reactions during estrogen therapy necessitate number of patients with atrophic vaginitis to pursue other alternative therapies. Lubricants and moisturizers can help keep vaginal secretion, increase comfort and so on. KY or Astronglide as lubricants can effectively alleviate short-term atrophic vaginitis symptoms, but the action can only last for a few minutes. These lubricants may flow all over the place, their vaginal retention is poor, and the patients often feel cold and discomfort upon the first application of these products.

A water-soluble polymer is often used as a carrier for drug delivery system. Bioadhesive drug delivery system utilizes the bioadhesive properties of water-soluble polymers to achieve targeted release of drugs at specific biological sites. Bioadhesion mainly relies on the nature of the bioadhesive agent. The first stage of bioadhesion starts with the intimate contact between the bioadhesive polymer and a biological surface; the second stage of bioadhesion involves the penetration of bioadhesive polymer into epithelial or mucosal tissue. Mucosa network at physiological pH carries negative charges, the high density of charges on the mucous membrane bearing sialic acid and sulfuric acid can significantly reinforce bioadhesion.

Bioadhesive polymers may be used in drug systems for the oral, ophthalmic, nasal, dermal, vaginal, and pulmonary delivery etc. Since these polymer materials have high viscosity, they are also widely used in controlled-release and sustained-release formulations. The sustained release formulations comprising of polycarbophil and chlorothiazide, for example, can be orally administered to rats to continuously release chlorothiazide for up to 8 hours. Polycarbophil offers a gastric retention function, which is attributable to its high viscosity. In one study, duodenal intubation technique was used to determine canine gastric emptying rate. The researchers found that the higher the concentration of polycarbophil, the longer the gastric emptying time. The conclusion was that polycarbophil increased gastric retention via its apparent viscosity. Since polycarbophil contains many carboxylic acid groups, it also has a high degree of pH buffering capacity. In addition, this polymeric material has water swellability. Therefore it has an extraordinary ability to absorb and retain moisture. As a result, it can be used as a water-soluble lubricant.

CN101773516B disclosed a vaginal gel using polycarbophil and carbomer as acidic agents, methylparaben, ethylparaben and propylparaben sodium salts as alkaline agents, and water and glycerin as solvents. The composition have a pH buffering capacity, playing a role of maintaining vaginal pH. CN102266283B disclosed a sterile vaginal gel containing polycarbophil and carbomer and V80 or quaternary ammonium compounds for the treatment of vaginitis. CN102688182A disclosed a vaginal pH buffered antimicrobial gel, and its main components are polycarbophil, carbomer, disodium edetate, chitosan, glycerin, triethanolamine, methyl p-hydroxybenzate and deionized water. The invention aimed to use the weak acid and chitosan antimicrobial properties to treat vaginitis. In spite of the use of polycarbophil and carbomer as common acidic buffering systems in the above patents, other antimicrobial or antibacterial ingredients varied. The above patents proposed the corresponding solutions for lowering vaginal pH and treatment of vaginitis, but none of them proposed a solution for the resistant vaginitis.

CN101951868B disclosed the use of boric acid as single active pharmaceutical ingredient for the treatment and/or prevention of vaginal infections and/or pathogenic vaginal biofilm. Although the in vitro experiments carried out in this patent proved the efficacy of boric acid on obliterating bacterial biofilm, it did not come up with a boric acid dosage form or formulation for clinical application, nor did it conceive a solution of how to use a practical boric acid formulation to treat ill patients with fungal or bacterial vaginitis.

### OBJECTIVES OF THE INVENTION

There is not any published literature reporting using bioadhesive polymer materials for the preparation of boric acid vaginal formulation. No vaginal adhesive boric acid formulations are ever used as antibacterial agents. The vaginal bioadhesive boric acid formulation disclosed by this patent can be applied for treating fungal, bacterial, and trichomonal vaginitis, especially the treatment of recurrent and resistant vaginitis. It may also has a role in regulating vaginal pH value, moisturizing and lubricating. As a result, it emerges to become a treatment option for atrophic vaginitis complicated or not complicated with bacterial or fungal or trichomoniasis infections. The invention is intended to replace the extemporaneous preparation of boric acid capsules and to provide doctors and patients more manageable, more efficient, more comfortable, more convenient solution for recurrent vaginitis.

Therefore, a main objective of the present invention is to provide a bioadhesive boric acid formulation that enables the maintenance of relative high boric acid concentration in vaginal fluid. Because boric acid at a concentration of 10-20 mg/mL inhibited almost all common bacterial or fungal growth, ideally vaginal bioadhesive boric acid preparation ought to maintain boric acid concentration at similar levels. The boric acid release characteristics can be simulated with in vitro experiments.

Another objective of this invention is to provide a vaginal bioadhesive boric acid formulation to inhibit bacterial and fungal growth. This inhibition efficacy can proved by in vitro methods.

Another objective of this invention is to provide a bioadhesive boric acid formulation that can be used for fungal, bacterial, and Trichomonas vaginitis, especially treatment of recurrent and resistant vaginitis.

Another objective of this invention is to provide a vaginal bioadhesive boric acid formulation to adjust vaginal pH, lubricate and moisturize vaginal tract, so that it may play a role in treating atrophic vaginitis complicated or not complicated with bacterial or fungal or trichomoniasis infections.

Another objective of this invention is to provide a vaginal bioadhesive boric acid formulation, including but not limited to, semi-solid preparations, such as gels, cream, cream, soft capsules, or other semi-solid dosage forms.

Another objective of this invention is to provide a vaginal bioadhesive boric acid formulation with uniform continuity such that there will be no grainy sensation and discomfort for the users.

Another objective of this invention is to provide a vaginal bioadhesive boric acid preparation that is safe to use, without irritation, sensitization, and cytotoxicity.

Another objective of this invention is to provide a preparation process for the vaginal bioadhesive boric acid formulation, where the process can enable quality products with uniform continuity, allow pilot scale-up, and produce sufficient quantity of such formulation for clinical research.

Another objective of this invention is to provide a method for treating patients with vaginal bioadhesive boric acid formulation that contains pharmaceutically acceptable boric acid, hydrate and other boric acid derivatives. Methods described herein is meant to give patients the vaginal bioadhesive boric acid formulations within the scope of effective dosage regimen. The general disease conditions include, but not limited to fungal, bacterial, trichomonal vaginitis, atrophic vaginitis, cervicitis, and other gynecological inflammations.

Other objectives, advantages and novelties of this invention will be demonstrated in the description of the invention section below. After examining this patent, those skilled in the art would be taught with the know-hows described herein. The findings derived from such teachings are also within the scope of this invention.

### BRIEF DESCRIPTION OF THE DRAWINGS

FIG. 1: Bioadhesive Boric Acid Gel Prepared in Lab Scale

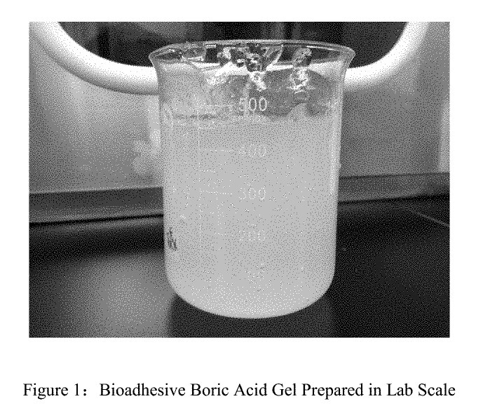

FIG. 2: Bioadhesive Boric Acid Gel Packaged in Vaginal Applicator

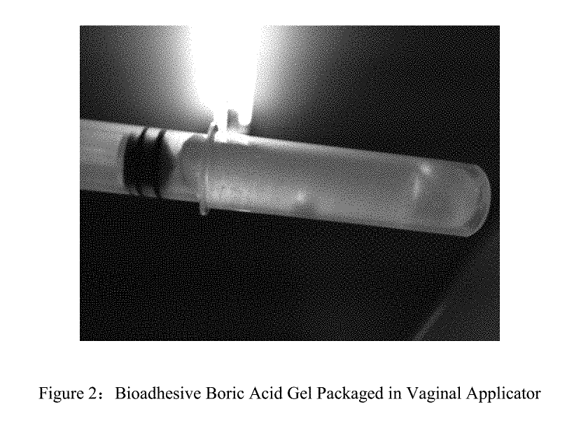

FIG. 3: Vaginal Boric Acid Retention and Release Study on Bioadhesive Boric Acid Gel

FIG. 4: Calibration Curve for Boric Acid Quantitation

FIG. 5: Boric Acid Release Profile of Bioadhesive Boric Acid Gel in Artificial Vaginal Fluid

FIG. 6: Antibacterial Potency of Bioadhesive Boric Acid Vaginal Gel

### DESCRIPTION OF THE INVENTION

### 1. Definition and Nomenclature

“Bioadhesive” Described herein means two materials (of which at least one is biological) can be held together under interfacial force after a long period of time. “Bioadhesive” means that the carrier systems carrying the drug can be interlocked with a specific biological surface, which may be the upper surface of epithelial tissue or mucosal tissues.

“Bioadhesive polymers” described herein are polymers materials that can serve as binders. They may be derived from a biological monomer, such as a synthetic form of sugar glue, or other synthetic glue designed to adhere to the biological tissues.

Bioadhesive polymer materials described herein include, but are not limited to, acacia gum, alginate, carbomer/carbopol, polycarbophil, hydroxypropyl methylcellulose, polycarbophil, hyaluronic acid, pectin, sodium hyaluronate, polyvinyl alcohol, polyvinyl pyrollidone, and tragacanth.

“Vaginal bioadhesive boric acid formulation” described herein refers to the formulation recipe that can be used to retain active ingredients such as boric acid in the vagina and slowly release the active ingredients over time. This said vaginal formulation plays an important role for vaginal drug delivery because vaginal fluid secretion often causes rapid loss of active ingredient. Such a formulation of the active ingredient can help to achieve and maintain a certain concentration of the active ingredient and control the release of the active ingredient as to achieve the desired therapeutic effect.

### 2. New Formulation Recipe

In one aspect, the present invention provides a vaginal boric acid formulation comprising of 0.1 to 10% of boric acid and bioadhesive polymers, can retain and release boric acid as antibacterial ingredient in the vaginal fluid.

In embodiment examples 11-16, the present invention provides bioadhesive vaginal preparations that contain both boric acid and bioadhesive polymers.

In a preferred embodiment, the carbomer 974 or polycarbophil act as retention and release matrix for the said vaginal formulations.

In embodiment examples 17-18, the present invention provides vaginal preparations that contain boric acid, polyacrylic acid polymers (Carbopol 974 and/or polycarbophil), and chitosan.

In a preferred embodiment, the vaginal formulations contain chitosan, for enhancing the bacteriostatic effect. Chitosan is a natural antibacterial agent and the concentration of higher than 0.01% of chitosan will produce grainy and course sensation, and affect the uniformity of the formulation.

In embodiment samples 1-3, the present invention provides vaginal preparations that combine boric acid and polyacrylic acid polymers (Carbopol 974 and/or polycarbophil).

In a preferred embodiment, 5% of boric acid is used as an active ingredient of such formulations. Such preparations contain boric acid concentration as high as possible, to enhance the antimicrobial effect of the formulation. However, more than 5% of boric acid is difficult to dissolve in water, causing the grainy and course sensation.

In a preferred embodiment, 1.5% polyacrylic polymer material is used as retention and release matrix in the formulation. Less than 0.5% of the polyacrylic polymer material formed relatively thin gels because the viscosity is too low. More than 3% of polyacrylic acid polymer material to form a relatively thick gel because viscosity is too high.

In a preferred embodiment, such a vaginal formulation containing glycerol, for increasing the moisturization and lubrication.

In a preferred embodiment, such a vaginal formulation contain methylparaben, propylparaben, and/or potassium sorbate as a preservative.

In embodiment examples 19-25, the present invention provides other semi-solid and solid vaginal preparations, including creams, ointments, and soft gelatin capsules. These preparations contain a combination of boric acid and polyacrylic acid polymer materials (Carbopol 974 and/or polycarbophil).

In embodiment example 9, the present invention provides production methods for a vaginal gel containing boric acid and polyacrylic acid polymer materials (Carbopol 974 and polycarbophil). Such boric formulation can be prepared in a uniform texture and on a large scale suited for clinical research and commercialization.

In a preferred embodiment, this production method requires that the boric acid be first dissolved in water.

In the preferred embodiment, this production method requires polymer material with bioadhesive properties be added to the aqueous solution portion wise with vigorous stirring.

In another aspect, the present invention provides an evaluation of such vaginal bioadhesive boric acid preparations. This vaginal boric acid formulation retains and release boric acid as antibacterial ingredient in vaginal fluid.

In embodiment examples 5-7, the present invention provides a formula for making simulated vaginal fluid, a method to construct artificial vagina by using a segment of pig vagina and a condenser with the temperature maintained at 37° C., and a method to utilize vaginal gel retention device containing artificial vagina to determine boric acid release profile.

In embodiment example 4, the present invention provides the use of methyl cellulose instead of polyacrylic acid polymer material (Carbopol 974 and polycarbophil) to prepare a boric acid gel, which is used as a control for demonstrating boric acid retention and release capabilities of polyacrylic polymer material (Carbopol 974 and polycarbophil).

In a preferred embodiment, comparing formulation I, II, III, and IV, using Carbopol 974 and polycarbophil simultaneously in preparing vaginal gel, results in a release of greater than 10 mg/ml of boric acid within 0-2 hours and greater than 2 mg/ml of boric acid thereafter in vaginal fluid. Carbomer 974 and/or polycarbophil used as vaginal gel matrix for boric acid formulation was superior to methyl cellulose, indicating that under the influence of bioadhesive polymer materials, boric acid can stay in the vagina for a prolonged time and the release of boric acid during the detention may enable the formulation to achieve the target antibacterial concentration.

In a preferred embodiment, the concentrations of Carbopol 974 and polycarbophil are 0.5% and 1% respectively.

### 3. Function and Usage

In one aspect, the present invention provides a vaginal bioadhesive formulation to maintain a high concentration of boric acid in the vaginal fluid over an extended period, for inhibiting the growth of bacteria and mold.

In embodiment example 8, the present invention provides in vitro experiments to evaluate the antibacterial activity of the vaginal bioadhesive boric acid preparation, Formulation I. Vaginal gel significantly inhibited and killed Pseudomonas aeruginosa, Staphylococcus aureus, Escherichia coli, and Candida albicans, demonstrating that vaginal preparations containing boric acid and polyacrylic acid polymer materials can effectively suppress bacteria and mold growth.

The said vaginal bioadhesive boric acid preparations can be used for the treatment of vaginal and cervical diseases, generally including but are not limited to fungal vaginitis, bacterial vaginosis, trichomoniasis, atrophic vaginitis, cervicitis, and other gynecological inflammation. It can also be used for relapsing fungal, bacterial, and trichomonas vaginitis that are resistant to other available treatments.

In another aspect, the present invention provides a vaginal bioadhesive boric acid formulation, regulating vaginal pH, moisturizing and lubricating vagina mucosa, as well as preventing and treating bacterial or fungal or trichomonas infection combined or not combined with atrophic vaginitis.

In embodiment example 10, the present invention provides the evaluations of appearance, sensory, physical, and chemical properties of Formulation I. The vaginal adhesive boric acid formulation has a pH between 2-5, in line with the natural physiological pH of the vagina. So it can be used for adjusting the vaginal pH and restoring the natural ability of vagina to resist the external invasion of bacteria. Formulation I has a uniform and smooth sensation and may play the role of lubricating atrophic vaginitis and ease clinical symptoms of atrophic vaginitis. Formulation I has a uniform and continuous texture, without causing any grainy and discomfort sensation.

In another aspect, the present invention provides a vaginal bioadhesive boric acid formulation with a good safety profile, causing no irritation, sensitization, and cytotoxicity.

In embodiment example 10, the present invention provides irritation, sensitization, and cytotoxicity tests on Formulation I and the experiments showed that Formulation I was not irritating, sensitizing, and cytotoxic.

In another aspect, the present invention provides a bioadhesive vaginal boric acid formulation, containing a pharmaceutically acceptable boric acid, its hydrates and other derivatives, for the treatment of patients with such formulation. The method administers vaginal boric acid formulation described above in the range of effective dosing regimen to treat gynecological diseases. The disease conditions in general include, but are not limited to, fungal vaginitis, bacterial vaginosis, trichomoniasis, atrophic vaginitis, cervicitis, and other gynecological inflammations.

Other objects, advantages, and novel features of the present invention are set forth in the above description. Those skilled in the art after reading this patent can understand the patent or learn by practice the true meaning and implications of this invention. For these aspects and other aspects not covered, if those skilled in the art after reading this patent could easily learn and apply the idea, concepts, techniques, know-hows, or knowledge taught by this patent without authorization, for product development, production, and marketing, such actions constitute infringement of the present invention.

### EXAMPLES

The following embodiment examples are used to further illustrate the invention. Embodiment examples are to provide the explanation for the invention, rather than to limit the invention. Therefore, within the scope of the claims of the invention, the simple alterations of the embodiment examples constitute patent infringement.

### Example 1

Preparation of Formulation I: Measured 83 ml purification water and heated to 55° C. Added 5 g boric acid, 0.2 g methylparaben and 0.03 g propylparaben while stirring the solution at a speed of 200-300 revolutions/minute for approximately one hour. Ensured the dissolution of solids and cooled to room temperature. Added 0.01 g chitosan and stirred for 15 minutes. Added 0.1 g potassium sorbate and 15 g glycerol and dissolved the solids by stirring for 5 minutes. Added 0.5 g carbopol 974 and 1 g polycarbophil and stirred for approximately 4 hours until a full swelling of the polymers.

### Example 2

Preparation of Formulation II: Measured 83 ml purification water and heated to 55° C. Added 5 g boric acid, 0.2 g methylparaben and 0.03 g propylparaben while stirring the solution at a speed of 200-300 revolutions/minute for approximately one hour. Ensured the dissolution of solids and cooled to room temperature. Added 0.01 g chitosan and stirred for 15 minutes. Added 0.1 g potassium sorbate and 15 g glycerol and dissolved the solids by stirring for 5 minutes. Added 1.5 g polycarbophil and stirred for approximately 4 hours until a full swelling of the polymers.

### Example 3

Preparation of Formulation III: Measured 83 ml purification water and heated to 55° C. Added 5 g boric acid, 0.2 g methylparaben and 0.03 g propylparaben while stirring the solution at a speed of 200-300 revolutions/minute for approximately one hour. Ensured the dissolution of solids and cooled to room temperature. Added 0.01 g chitosan and stirred for 15 minutes. Added 0.1 g potassium sorbate and 15 g glycerol and dissolved the solids by stirring for 5 minutes. Added 1.5 g carbopol 974 and stirred for approximately 4 hours until a full swelling of the polymers.

### Example 4

Preparation of Formulation IV as control: Measured 83 ml purification water and heated to 55° C. Added 5 g boric acid, 0.2 g methylparaben and 0.03 g propylparaben while stirring the solution at a speed of 200-300 revolutions/minute for approximately one hour. Ensured the dissolution of solids and cooled to room temperature. Added 0.01 g chitosan and stirred for 15 minutes. Added 0.1 g potassium sorbate and 15 g glycerol and dissolved the solids by stirring for 5 minutes. Added 1.5 g methylcellulose and stirred for approximately 4 hours until a full swelling of the polymers.

### Example 5

Prepare simulated vaginal fluid according to the following formula. Adjust the pH value to 4.2 in the end.

| Composition | Simulated vaginal fluid (g/L) |
|---|---|
| Sodium chloride | 3.51 |
| Potassium hydroxide | 1.40 |
| Calcium hydroxide | 0.222 |
| Bovine serum albumin | 0.018 |
| Lactic acid | 2.00 |
| Acetic acid | 1.00 |
| Glycerol | 0.16 |
| Urea | 0.40 |
| Glucose | 5.00 |
| pH value | 4.2 |

### Example 6

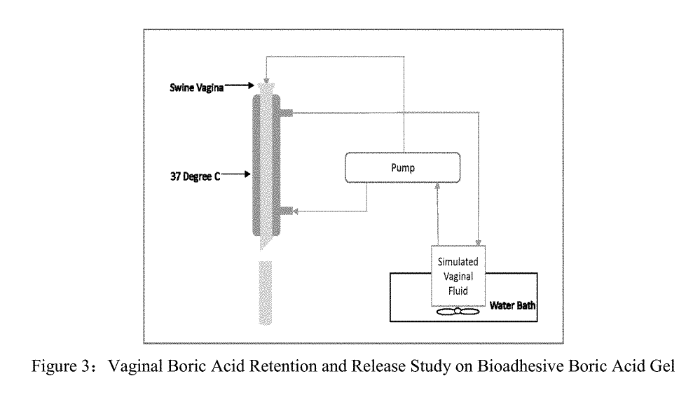

Took the pig's vagina (procured from farm and kept at 4° C.). Mounted the vagina to a condenser that matched the dimensions of the vagina per FIG. 3 (for example, length of the condenser is 11 cm, outer diameter 5 cm, inner diameter 1 to 2.4 cm). Evenly coated 3 g vaginal gel on the inner wall of the vagina. Added simulated vaginal fluid into a beaker. Infused simulated vaginal fluid preheated with a 37° C. water bath via a low speed pump at a rate of 3 ml/hour into the condenser attached with the pig's vagina. Meanwhile, pumped vaginal fluid into the sleeve jacket of the condenser to maintain 37° C., simulating human vaginal condition.

Collected simulated vaginal fluid off the condenser attached with the pig's vagina vaginal fluid with a small tube per sampling time points at 0-10 minutes, 10-20 minutes, 20-30 minutes 50-60 minutes 110-120 minutes 230-240 minutes 470-480 minutes, and 950-960-minute. Collected approximately 0.5 ml per tube. Determined the boric acid content after dilution of the collected vaginal fluid.

Compared the vaginal 5% boric acid gel with carbomer and polycarbophil (Formulation I), with polycarbophil only (Formulation II), with carbomer only (Formulation III), and methyl cellulose only (control group) in accordance with the above boric acid retention and release testing methods.

### Example 7

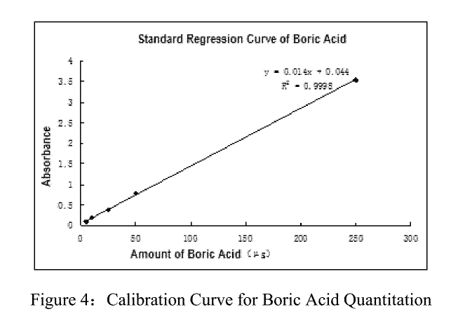

Accurately weighed 502.4 mg (purity 99.5%) boric acid into 100 ml volumetric flask and diluted to volume. The solution was diluted in series to achieve target concentrations for linearity standards. Took 1 mL of linearity standards that are equivalent to the amounts of 5 μg, 10 μg, 25 μg, 50 μg, and 250 μg boric acid respectively for constructing standard curve. Further derivatized boric acids per the following method and determined UV absorbance using UV-visible spectrophotometer. Plotted calibration curve per UV absorbance. Results are shown in FIG. 4.

Diluted the vaginal fluid collected off the condenser attached with pig's vagina to 250 ml. Pipetted 1 ml out for the colorimetric determination of boric acid content with cucurmin derivatization.

In particular, for the standard and sample, quantitative pipetted a certain volume (0 to 5 ml) of solution into 25 mL plastic tube and diluted with water to 5 ml. Added sulphuric acid (1+1) solution 1 ml, vortexed and mixed well. Then added 5 ml of ethyl hexanediol/chloroform (1:9) solution, sealed with the lid, and shook for about 2 minutes. Waited until the separation of two layers. Transferred the lower ethyl hexanediol/chloroform solution and filtered through 7 cm diameter filter paper. Transferred 1 ml of the filtrate into 50 mL plastic tubes. Added 1 ml of 0.1% cucurmin/glacial acetic acid (w/v) solution and then 0.5 ml of concentrated sulphuric acid, shook and let stand for 30 minutes. Added 25 ml of anhydrous ethanol and let stand for 10 minutes. Performed colorimetric determination of the absorbance at 550 nm with 1 cm curvet.

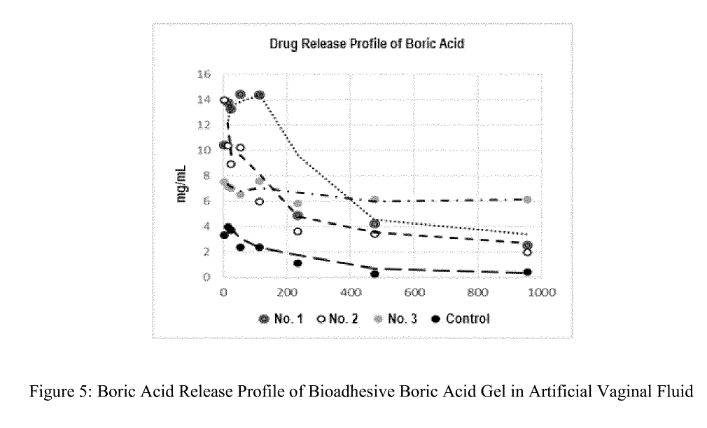

Results are shown in FIG. 5 and the tables below.

### Formulation I

| Tube number | 1 | 2 | 3 | 4 | 5 | 6 | 7 | 8 |
|---|---|---|---|---|---|---|---|---|
| Absorbance | 0.3357 | 0.4306 | 0.4165 | 0.4475 | 0.4464 | 0.1802 | 0.1630 | 0.1140 |
| Boric acid (mg) | 5.21 | 6.90 | 6.65 | 7.21 | 7.19 | 2.43 | 2.12 | 1.25 |

### Formulation II

| Tube number | 1 | 2 | 3 | 4 | 5 | 6 | 7 | 8 |
|---|---|---|---|---|---|---|---|---|
| Absorbance | 0.4361 | 0.3341 | 0.2942 | 0.3301 | 0.2115 | 0.1457 | 0.1405 | 0.0993 |
| Boric acid (mg) | 7.00 | 5.18 | 4.47 | 5.11 | 2.99 | 1.82 | 1.72 | 0.99 |

### Formulation III

| Tube number | 1 | 2 | 3 | 4 | 5 | 6 | 7 | 8 |
|---|---|---|---|---|---|---|---|---|
| Absorbance | 0.2538 | 0.2450 | 0.2408 | 0.2270 | 0.2555 | 0.2067 | 0.2158 | 0.2154 |
| Boric acid (mg) | 3.75 | 3.59 | 3.51 | 3.27 | 3.78 | 2.91 | 3.07 | 3.06 |

### Formulation IV

| Tube number | 1 | 2 | 3 | 4 | 5 | 6 | 7 | 8 |
|---|---|---|---|---|---|---|---|---|
| Absorbance | 0.1362 | 0.1547 | 0.1486 | 0.1100 | 0.1099 | 0.0755 | 0.0515 | 0.0550 |
| Boric acid (mg) | 1.65 | 1.98 | 1.87 | 1.18 | 1.18 | 0.56 | 0.13 | 0.20 |

### Example 8

The method to culture fresh yeast is shown in the table below. Pseudomonas aeruginosa, Staphylococcus aureus, Escherichia coli, and Candida albicans are liquid cultures. So the bacteria were collected by centrifugation, washed with sterile solution of 0.9% sodium chloride and made a suspension of bacteria of about 108 cfu. Agar plate method was used for the determination of the number of bacteria contained in 1 ml bacterial suspension.

The liquid preparation of bacteria was placed at room temperature and should be used within 2 hours or used within 24 hours if stored at 2˜8° C.

| | | Culture | |
|---|---|---|---|
| Test strains | Test medium | Temperature | Culture Time |
| Staphylococcus aureus | Tryptic soy broth | 30~35° C. | 18~24 hours |
| (CMCC(B)26 003) | | | |
| Pseudomonas | Tryptic soy broth | 30~35° C. | 18~24 hours |
| aeruginosa | | | |
| (CMCC(B)10 104) | | | |
| Escherichia coli | Tryptic soy broth | 30~35° C. | 18~24 hours |
| (CMCC(B)44102) | | | |
| Candida albicans | Saouraud Dextrose | 20~25° C. | 24~48 hours |
| (CMCC(F) 98 001) | Broth | | |

Directly inoculated four types of bacteria as shown in above table into separate intact packages for testing. For each sample (about 3 grams), the inoculation volume was 20 μl with an inoculation density of 105˜106 cfu/grams. Inoculated bacteria such that the distribution of bacteria was even by mixing thoroughly. Then stored at 20˜25° C. while kept it away from light. Each time took out approximately 1 g from the package for testing.

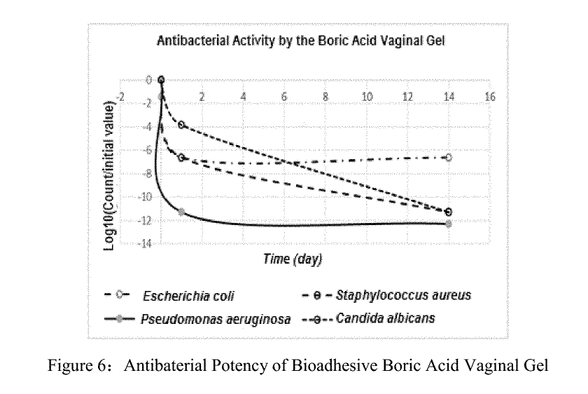

At 0, 14, and 28 days, diluted the content taken out of from the package 1000 times and used agar plate method for the determination of bacterial number. The results were shown in the following table and FIG. 6.

| | | Bacteria count (×1000) | | | |
|---|---|---|---|---|---|
| | | | Staphylococcus | Pseudomonas | |
| Test Article | Repeat | Escherichia coli | aureus | aeruginosa | Candida albicans |
| Bioadhesive | 1 | 1 | 1 | 0 | 285 |
| Boric Acid | 2 | 0 | 0 | 0 | 310 |
| Gel Day 0 | | | | | |
| Bioadhesive | 1 | 0 | 0 | 0 | 0 |
| Boric Acid | 2 | 1 | 0 | 0 | 0 |
| Gel Day 14 | | | | | |
| Bioadhesive | 1 | 0 | 0 | 0 | 0 |
| Boric Acid | 2 | 0 | 0 | 0 | 0 |
| Gel Day 28 | | | | | |
| Positive | 1 | ~200-2000 | ~200-2000 | ~200-2000 | ~200-2000 |
| Control Day 0 | 2 | ~200-2000 | ~200-2000 | ~200-2000 | ~200-2000 |

### Example 9

As an example, to produce 8 L of Formulation I:

Charged 6640 g purified water to the emulsifying mixer. Used the weighing bag or weighing flask to add 400 g of boric acid, 16 g of methylparaben, and 2.4 g of propylparaben into the mixer. Set the heating control panel and heated the mixture to 55° C. Started stirring at 50 Hz for 60 minutes until the full dissolution of the solids.

Cooled to below 40° C. Opened the lid and added 0.8 g chitosan. Closed the mixer and started stirring at 50 Hz for 30 minutes.

Opened the lid and added 8 g of sorbic acid and 1200 g of glycerin. Closed the mixer and stirred at 50 Hz for 5 minutes.

Opened the lid and added 40 g carbopol 974P NF which was filtered through 20 mesh sieve. Closed the mixer and started stirring at 50 Hz. When the powder was all added into the water, pumped down to vacuum until the pressure gauge reading was approximately 0.08 MPa. Stirred for 120 minutes until the full swelling of carbopol 974P NF.

Two hours later, opened the lid and added 80 g of polycarbophil through 20 mesh sieve. Closed the mixer and started stirring at 50 Hz. When the powder was all added into the water, pumped down to vacuum until the pressure gauge reading was approximately 0.08 MPa. Stirred for 120 minutes until the full swelling of polycarbophil AA-1.

Filled the product into the vaginal applicator after the stirring was complete. First used purified water and 75% of medical alcohol to clean and disinfect the gel filling apparatus to ensure the sanitation of the hopper and pipe. Adjusted the pressure regulator until the pressure gauge reading was 0.4 MPa and turned the metering valve to the desired filling quantity (3 g per applicator). Rotated the operation switch to manual mode. Lined up the feeding inlet of the vaginal applicator and loading outlet of the filling apparatus. Stepped on the foot switch on the filling apparatus and filled the gel product into the vaginal applicator. Continued with the filling while controlling the filling quantity.

Sealed the applicator after filling. Labeled and packaged into secondary product box and crate.

### Example 10

Assessed the appearance, physical properties (viscosity), chemistry (pH value), and safety (cytotoxicity, delayed hypersensitivity, and vaginal irritation) of the bioadhesive boric acid gel, results are shown as follows:

| Test Items | Acceptance criteria | Results |
|---|---|---|
| Appearance | Transparent or white semisolid, | Conforms |
| | uniform and fine, stable as a gel | |
| | without drying or liquefying at | |
| | room temperature | |
| Dynamic viscosity | Not less than 1000 mPa · s | 130370 |
| pH value | pH value 2-5 | 3.4 |
| Delayed hypersensitivity | not more than level 2 | Level 1 |
| Delayed hypersensitivity | No delayed hypersensitivity | Conforms |
| | reaction | |
| Vaginal irritation | No vaginal irritation reaction | Conforms |

### Example 11

Measured 83 ml purified water. Heated it to 55° C. While stirring and mixing (speed of 200-300 revolutions/minute), added 5 g boric acid. Stirred for approximately 1 hour. Cooled to room temperature. Added 15 g glycerol and stirred for 5 minutes. Added 1.5 g polycarbophil and stirred for approximately 4 hours until the full swelling of polycarbophil.

### Example 12

Measured 83 ml purified water. Heated it to 55° C. While stirring and mixing (speed of 200-300 revolutions/minute), added 5 g boric acid. Stirred for approximately 1 hour. Cooled to room temperature. Added 15 g glycerol and stirred for 5 minutes. Added 1.5 g Carbopol 934, and stirred for approximately 4 hours until the full swelling of Carbopol 934.

### Example 13

Measured 83 ml purified water. Heated it to 55° C. While stirring and mixing (speed of 200-300 revolutions/minute), added 5 g boric acid. Stirred for approximately 1 hour. Cooled to room temperature. Added 15 g glycerol and stirred for 5 minutes. Added 1.5 g Carbopol 940, and stirred for approximately 4 hours until the full swelling of Carbopol 940.

### Example 14

Measured 83 ml purified water. Heated it to 55° C. While stirring and mixing (speed of 200-300 revolutions/minute), added 5 g boric acid. Stirred for approximately 1 hour. Cooled to room temperature. Added 15 g glycerol and stirred for 5 minutes. Added 1.5 g Carbopol 974, and stirred for approximately 4 hours until the full swelling of Carbopol 974.

### Example 15

Measured 83 ml purified water. Heated it to 55° C. While stirring and mixing (speed of 200-300 revolutions/minute), added 5 g boric acid. Stirred for approximately 1 hour. Cooled to room temperature. Added 15 g glycerol and stirred for 5 minutes. Added 1.5 g polyvinyl pyrrolidone, and stirred for approximately 4 hours until the full swelling of polyvinyl pyrrolidone.

### Example 16

Measured 83 ml purified water. Heated it to 55° C. While stirring and mixing (speed of 200-300 revolutions/minute), added 5 g boric acid. Stirred for approximately 1 hour. Cooled to room temperature. Added 15 g glycerol and stirred for 5 minutes. Added 1.5 g tragacanth gum, and stirred for approximately 4 hours until the full swelling of tragacanth gum.

### Example 17

Measured 83 ml purified water. Heated it to 55° C. While stirring and mixing (speed of 200-300 revolutions/minute), added 5 g boric acid. Stirred for approximately 1 hour. Cooled to room temperature. Added 0.01 g chitosan and stirred for 15 minutes. Added 15 g glycerol and stirred for 5 minutes. Added 1.5 g polycarbophil, and stirred for approximately 4 hours until the full swelling of polycarbophil.

### Example 18

Measured 83 ml purified water. Heated it to 55° C. While stirring and mixing (speed of 200-300 revolutions/minute), added 5 g boric acid. Stirred for approximately 1 hour. Cooled to room temperature. Added 0.01 g chitosan and stirred for 15 minutes. Added 0.1 g potassium sorbate and then 15 g glycerol and stirred for 5 minutes. Added 1.5 g polycarbophil, and stirred for approximately 4 hours until the full swelling of polycarbophil.

### Example 19

Measured 83 ml lotion preparation (such as fragrance-free JERGENS lotion on the market). Heated it to 55° C. While stirring and mixing (speed of 200-300 revolutions/minute), added 5 g boric acid. Stirred for approximately 1 hour. Added 0.5 g polycarbophil and cooled to room temperature. Stirred for approximately 1 hours until a uniform lotion was obtained.

### Example 20

Measured 83 ml purified water. Heated it to 55° C. While stirring and mixing (speed of 200-300 revolutions/minute), added 5 g boric acid. Stirred for approximately 1 hour. Cooled to room temperature. Added 0.01 g chitosan and stirred for 15 minutes. Added 0.1 g potassium sorbate and then 15 g glycerol and stirred for 5 minutes. Added 1.5 g polycarbophil, and stirred for approximately 4 hours until the full swelling of polycarbophil. Pour the gel onto the tampons used for the mucous membrane.

### Example 21

Weighed approximately 20 g of PEG800, 60 g of PEG4000, heated to 80° C. While stirring and mixing (speed of 200-300 revolutions/minute), added 5 g boric acid. Stirred for approximately 1 hour. Transferred to the suppository mold. Cooled to room temperature, or cooled in the refrigerator until solidified, then ejected the suppository for packaging.

### Example 22

Measured 83 ml purified water. Heated it to 55° C. While stirring and mixing (speed of 200-300 revolutions/minute), added 5 g boric acid. Stirred for approximately 1 hour. Cooled to room temperature. Added 0.01 g chitosan and stirred for 15 minutes. Added 0.1 g potassium sorbate and then 15 g glycerol and stirred for 5 minutes. Added 0.5 g polycarbophil. Meanwhile, added 1.5 g bee wax into 10 ml mineral oil. Added 2 g glycerol monostearate, 0.2 g propylparaben and heated it to 55° C. Mixed aqueous and oil phases for approximately 4 hours until a uniform emulsion is obtained.

### Example 23

Weighed approximately 5 g of boric acid, 0.01 g of chitosan, and 1.5 g of polycarbophil. Mixed the above for approximately 1 hour until a uniform powder is obtained. The powder can be used directly for vaginal application, or poured into a capsule for vaginal application.

### Example 24

Weighed approximately 5 g of boric acid, 0.01 g of chitosan, 5 g of microcrystalline cellulose, 10 g of lactose, 0.5 g of sodium carboxymethylcellulose, 0.1 g of magnesium stearate, and 1.5 g of polycarbophil. Mixed for approximately 1 hour until a uniform powder is obtained. The powder was then punched into tablet with tablet compression machine for vaginal application.

### Example 25

Measured 30 ml of PEG400, 50 ml of propylene glycol, 10 ml of glycerol medium chain triester, and 1 ml of polysorbate. Heated the mixture to 55° C. While stirring and mixing (speed of 200-300 revolutions/minute), added 5 g boric acid. Stirred for approximately 1 hour. Cooled to room temperature. Added 0.01 g chitosan and stirred for 15 minutes. Added 0.1 g potassium sorbate and 1.5 g polycarbophil. Stirred for approximately 4 hours until the full swelling of the polymer and a uniform semisolid was obtained. Filled the semisolid into soft gel for vaginal application.

1. A bioadhesive boric acid formulation, consisting of 0.1-10% boric acid and bioadhesive polymers, for retaining and releasing antibacterial ingredient boric acid;

2. The formulation according to claim 1, wherein the formulation contains polyacrylic acid and related polymers, such as carbopol and/or polycarbophil, as bioadhesive polymers;

3. The formulation according to claim 2, wherein the formulation contains chitosan, or other antibacterial agents, for enhancing the antibacterial action of the formulation;

4. The formulation according to claim 2, wherein the formulation contains glycerin, or other moisturizing agents, for improving moisture-keeping and lubrication actions of the formulation;

5. The formulation according to claim 2, wherein the formulation contains methylparaben, propylparaben, and/or potassium sorbate, or other antimicrobial agents, as preservatives;

6. The formulation according to claim 2, wherein the formulation is semisolid or solid preparations, such as gel, cream, paste, soft capsule, hard capsule, or other semisolid and solid formulations;

7. The formulation according to claim 1, wherein during the preparation of the said formulation, boric acid is dissolved in water at the beginning of the process;

8. The formulation according to claim 1, wherein during the preparation of the said formulation, the bioadhesive polymers need to be added portion-wise into the water solution with vigorous stirring;

9. The use of the formulation according to claim 1 to treat vaginal and cervical diseases, including but not limited to fungal, bacterial, trichomoniasis, atrophic vaginitis, cervicitis, and other gynecological inflammation diseases.
